(19) United States
(12) Patent Application Publication
(10) Pub. No.: US 2020/0330045 A1
(43) Pub. Date: Oct. 22, 2020

(54) METHOD AND SYSTEM FOR ANALYZING RISK ASSOCIATED WITH RESPIRATORY SOUNDS

(71) Applicant: SALCIT TECHNOLOGIES PRIVATE LIMITED, Hyderabad (IN)

(72) Inventors: Narayana Rao VENKATA NAGA SRIPADA, Hyderabad (IN); Naveen Kumar VODNALA, Rangareddy (IN); Padma Shayi Shayi YARLAGADDA, Hyderabad (IN); Baswaraj MAMIDGI, Telangana (IN)

(73) Assignee: SALCIT TECHNOLOGIES PRIVATE LIMITED, Hyderabad (IN)

(21) Appl. No.: 16/768,872

(22) PCT Filed: Nov. 13, 2018
(86) PCT No.: PCT/IN2018/050745

§ 371 Date: Jun. 1, 2020

(30) Foreign Application Priority Data
Dec. 14, 2017 (IN) 201741045066

(51) Int. Cl. A61B 5/00; A61B 7/00; A61B 7/02; G16H 50/30; G16H 40/67

(52) U.S. Cl. CPC A61B 5/7275; A61B 7/003; A61B 7/026; A61B 5/01; A61B 5/0004; G16H 50/30; G16H 40/67; A61B 5/7475

(57) ABSTRACT

Embodiments of the present disclosure relates to a method and system for analyzing risk associated with respiratory sounds. The system comprises a respiratory monitoring device to assign risk category to a plurality of respiratory sound signals captured by at least one acoustic sensor. The present disclosure includes receiving the respiratory sound signals captured by at least one acoustic sensor and a user input data comprising information related to symptoms from a user interface of a user device. The present disclosure further includes deriving primary respiratory sound characteristics for each captured respiratory sound signal, and determining secondary respiratory sound characteristics based on the primary respiratory sound characteristics. The presence of inflammation in one or more of airway, pleura and parenchyma is determined based on the secondary respiratory sound characteristics, and a risk category associated with respiratory sound signal is assigned based on the presence of inflammation and the user input data.

### TECHNICAL FIELD

The present subject matter is related, in general to health advisory and more particularly, but not exclusively to a method and system for analyzing risk associated with respiratory sounds.

### BACKGROUND

Unusual respiratory sounds are the most common and primary symptom for several respiratory diseases. Respiratory sounds such as cough sounds, wheeze sounds and breathing sounds carry vital information on the state of the respiratory system. Respiratory sounds will help in understanding the inflammation in respiratory system including airways, pleura and parenchyma. Being a very often ignored symptom, the cough or wheeze can sometimes herald a chronic, fatal, debilitating condition. Analyzing the risk associated with respiratory sounds may help in understanding the health condition and encourage the patient to seek early treatment. Some of the existing devices use methods that can detect only the respiratory sounds from the sound recordings but unable to accurately identify risk associated with respiratory sounds. Some other methods include detection of risk involved in respiratory sounds but fails to determine the risk category i.e. one of high risk, low risk or moderate risk, and also fails in accurate determination as the parameters used for determination are limited. However, as the major goal is risk stratification, there is no proper methodology to analyze risk involved in respiratory system. Therefore, there is a need for a method and system for analyzing risk associated with respiratory sounds.

### SUMMARY

One or more shortcomings of the prior art are overcome, and additional advantages are provided through the present disclosure. Additional features and advantages are realized through the techniques of the present disclosure. Other embodiments and aspects of the disclosure are described in detail herein and are considered a part of the claimed disclosure.

In an aspect of the present disclosure a method of analyzing risk associated with respiratory sounds, is disclosed. The method comprises receiving respiratory sound signals that are captured by acoustic sensors, and a user input data from user interface of a user device. The method includes deriving respiratory sound characteristics for each captured respiratory sound signal. Further, the method includes determining presence of inflammation in airway part, parenchyma and pleura based on the determined respiratory sound characteristics. Upon determining the presence of inflammation, a risk category is assigned to each respiratory sound signal based on presence of inflammation and the user input data comprising information associated with chest related symptoms and other generic symptoms.

Further, the present disclosure relates to a system for analyzing risk associated with respiratory sounds. The system comprises acoustic sensors to capture respiratory sound signals and a user device coupled with the acoustic sensors to receive user input data from user. The user device transmits the captured respiratory sound signals and the user input data to a respiratory monitoring device. The respiratory monitoring device (RMD) comprises a processor configured to receive the respiratory sound signals and the user input data. Further, the processor is configured to derive respiratory sound characteristics for each respiratory sound signal. Also, the processor is configured to determine presence of inflammation in airway part, parenchyma and pleura based on the determined respiratory sound characteristics. Further, the processor is configured to assign risk category for each respiratory sound signal based on the presence of inflammation and the user input data comprising information associated with chest related symptoms and other generic symptoms.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 depicts an exemplary architecture of a system for analyzing risk associated with respiratory sounds in accordance with some embodiments of the present disclosure;

FIG. 2 is a block diagram illustration of respiratory monitoring device of FIG. 1 in accordance with some embodiments of the present disclosure;

FIG. 3a depicts a flowchart showing a method for analyzing risk associated with respiratory sounds in accordance with some embodiments of the present disclosure;

FIG. 3b shows an exemplary screenshot of a user device enabling user to provide user input data in accordance with some embodiments of the present disclosure;

FIG. 3c shows an exemplary screenshot of user device like mobile device illustrating analysis of risk in accordance with some embodiments of the present disclosure;

FIG. 3d shows the analysis result of FIG. 3c using the proposed framework in accordance with some embodiments of the present disclosure; and

FIG. 4 is a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the scope of the disclosure.

The terms “comprises”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure relates to a method and system for analyzing risk associated with respiratory sounds. The method includes receiving a plurality of respiratory sound signals captured by at least one acoustic sensor, and a user input data from a user interface of a user device coupled with a respiratory monitoring device. The method further includes deriving one or more primary respiratory sound characteristics including Pitch, Log energy, Zero crossings, Mel-frequency cepstral coefficients (MFCC) and Formant frequencies for each captured respiratory sound signal. The method also includes determining one or more secondary respiratory sound characteristics for each respiratory sound signal based on the primary respiratory sound characteristics. Upon determining the respiratory sound characteristics, presence of inflammation in one or more of airway part, pleura and parenchyma is determined. Based on the presence of inflammation thus determined, a risk category associated with each respiratory sound signal is assigned accordingly. The secondary respiratory sound characteristics determined from the captured respiratory sound signals are used in risk analysis, and the risk associated with respiratory sounds are effectively categorized. Thus, the proposed disclosure eliminates the complexity of risk analysis.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

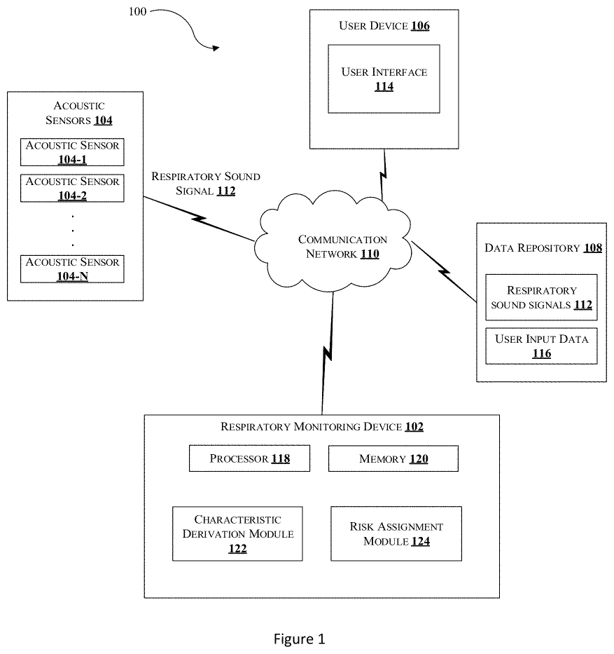

FIG. 1 illustrates an exemplary architecture of a system for analyzing risk associated with respiratory sounds in accordance with some embodiments of the present disclosure.

As shown in FIG. 1, the exemplary system 100 comprises one or more components configured for analyzing risk associated with respiratory sounds. In one embodiment, the exemplary system 100 comprises a respiratory monitoring device 102 (hereinafter referred to as RMD), one or more acoustic sensors 104-1, 104-2 . . . 104-N (hereinafter collectively referred to as acoustic sensor 104), a user device 106, a data repository 108 communicatively coupled via a communication network 110. The communication network 110 may include, without limitation, a direct interconnection, local area network (LAN), wide area network (WAN), wireless network (e.g., using Wireless Application Protocol), the Internet, etc.

The acoustic sensor 104 may be for example, an audio recorder or a microphone placed nearby to a person or a patient to record respiratory sounds. In one embodiment, the acoustic sensor 104 may be a microphone or audio recorder of user device 106 enabled to capture respiratory sound signals 112 of the person or the patient. The acoustic sensor 104 captures respiratory sound signals 112 including for example, cough sound signals, wheeze sound signals and breathing sound signals. The respiratory sound signals 112 captured by the acoustic sensor 104 may be stored in the data repository 108 coupled with the RMD 102.

The user device 106 may be a mobile device generally a portable computer or a computing device including the functionality for communicating over the communication network 110. For example, the mobile device can be a conventional web-enabled personal computer in the home, mobile computer (laptop, notebook, or subnotebook), Smart Phone (iPhone, Android), tablet computer or another device capable of communicating over the Internet or other appropriate communications network. In one embodiment, the user device 106 comprises a user interface 114 to receive a user input data 116 from at least one user. In another embodiment, the user device 106 is coupled with the acoustic sensor 104. The user device 106 is configured to transmit the respiratory sound signals 112 captured by the acoustic sensor 104 and the user input data 116 to the RMD 102.

The data repository 108 stores a plurality of respiratory sound signals 112 captured by the acoustic sensor 104. In one embodiment, the data repository 108 is configured to store the plurality of respiratory sound signals 112 of the person or the patient received from the user device 106. For example, the respiratory sound signals 112 may include one of cough sound signals, wheeze sound signals and other breathing sound signals. In another embodiment, the data repository 108 is also configured to store the user input data 116 received from the user interface 114 of the user device 106. For example, the user input data 116 may comprise information associated with chest related symptoms including Shortness of Breath, chest pain, Hemoptysis and other generic symptoms including body temperature of the person or the patient i.e. fever, Fatigue, Loss of Appetite. The data repository 108 may be integrated with RMD 102, in one embodiment. In another embodiment, the data repository 108 may be configured as a standalone device independent of RMD 102.

The RMD 102 comprises at least a processor 118 and a memory 120 coupled with the processor 118. The processor 118 may be for example, any processing unit capable of processing acoustic signals and the user input data 116. The RMD 102 further comprises an acoustic characteristics derivation module 122 and a risk assignment module 124. The characteristics derivation module 122 is configured to derive primary respiratory sound characteristics of the respiratory sound signals 112 captured by the acoustic sensor 104. The characteristics derivation module 122 is further configured to determine secondary respiratory sound characteristics based on the primary respiratory sound characteristics. The risk assignment module 124 is configured to determine presence of inflammation in one or more of smaller airway, larger airway, parenchyma and pleura based on the determined secondary respiratory sound characteristics. The risk assignment module 124 is further configured to assign a risk category associated with each respiratory sound signal based on the determined presence of inflammation and the user input data 116 comprising symptoms.

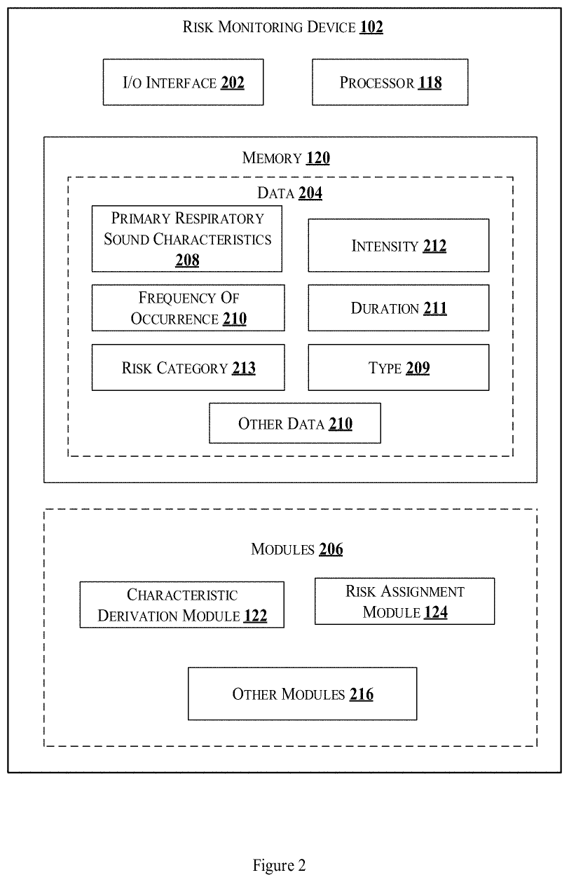

In an embodiment, the RMD 102 may be a typical RMD as illustrated in FIG. 2. The RMD 102 comprises the processor 118, the memory 120, and an I/O interface 202 communicatively coupled with the processor 118. The RMD 102 further includes data 204 and modules 206. In one implementation, the data 204 may be stored within the memory 116. In some embodiments, the data 204 may be stored within the memory in the form of various data structures. Additionally, the data 204 may be organized using data models, such as relational or hierarchical data models. In one example, the data 204 may include the primary respiratory sound characteristics 208, the secondary respiratory sound characteristics including type 209, frequency of occurrence 210, duration 211 and intensity 212 of the respiratory sound signals 112, a risk category 213 and other data 214. In one example, the risk category 213 defines the measure of risk or risk level involved or associated with captured respiratory sound signal 112, and the risk category 213 may include one of high risk, moderate risk, low risk and negligible risk. The other data 214 may store data, including temporary data and temporary files, generated by the modules 206 for performing the various functions of the risk monitoring device 102.

The modules 206 may include, for example, the characteristics derivation module 122 and the risk assignment module 124. The modules 206 may also comprise other modules 216 to perform various miscellaneous functionalities of the RMD 102. It will be appreciated that such aforementioned modules may be represented as a single module or a combination of different modules. The modules 206 may be implemented in the form of software executed by a processor, hardware and/or firmware.

In operation, the acoustic sensors 104 captures or records plurality of respiratory sound signals 112. In one example, the user device 106 coupled with the RMD 102 receives the respiratory sound signals 112 captured by the acoustic sensors 104, and the user input data 116 from the user, and transmits to the RMD 102. The RMD 102 processes the received respiratory sound signals 112 to analyze the risk associated with the respiratory sounds. In one embodiment, the characteristic derivation module 122 derives the primary respiratory sound characteristics 208 for each captured respiratory sound signal 112. For example, the primary respiratory sound characteristics 208 may include Pitch of the acoustic signal, Log energy, Zero crossings, Mel-frequency cepstral coefficients (MFCC) and Formant frequencies of the respiratory sound signal 112. The characteristic derivation module 122 further determines secondary respiratory sound characteristics based on the primary respiratory sound characteristics 208.

In one embodiment, the secondary respiratory sound characteristics include the frequency of occurrence 210, the duration 211, the intensity 212 of the respiratory sound signal 112 and type 209 of the respiratory sound signal. In one example, the frequency of occurrence 210 may be defined as the number of respiratory sounds i.e. cough sounds or wheeze sounds occurred in specified time period. The duration 211 may be defined as the length of time that the respiratory sound exists. The intensity 212 may be defined as the measure of loudness of the captured respiratory sound signals 112. The risk assignment module 124 assigns the risk category 213 associated with each respiratory sound signal 112. In one embodiment, the risk assignment module 124 determines the presence of inflammation in one or more of smaller airway, larger airway, parenchyma and pleura based on the determined secondary respiratory sound characteristics. In one example, the presence of inflammation may indicate the unusual functionality of any of the part of the respiratory system i.e. the airway part, parenchyma and pleura. Further, the risk assignment module 124 assigns a risk category 213 associated with each respiratory sound signal 112 based on the presence of inflammation and the user input data 116 comprising symptoms, wherein the risk category 213 may be one of high risk, moderate risk, low risk and negligible risk.

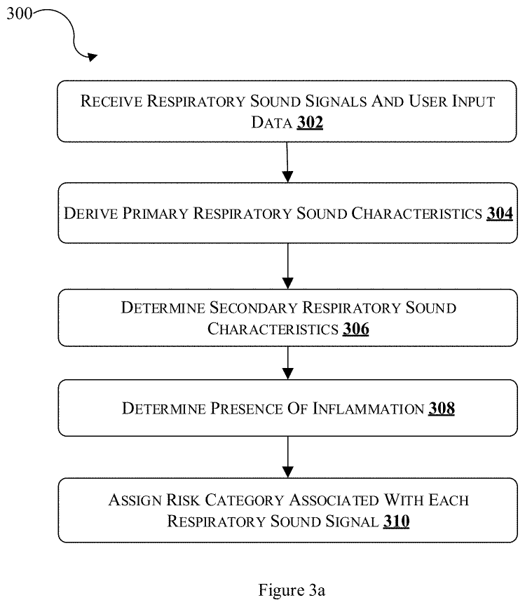

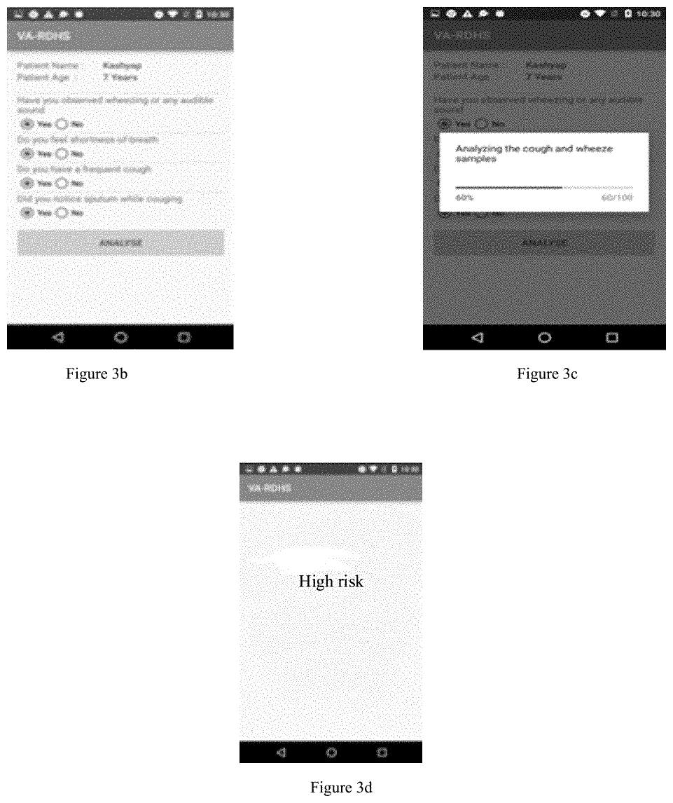

FIG. 3a illustrates a flowchart showing a method for analyzing risk associated with respiratory sounds in accordance with some embodiments of the present disclosure.

As illustrated in FIG. 3a, the method 300 comprises one or more blocks implemented by the processor for analyzing risk associated with respiratory sound signals using RMD 102. The method 300 may be described in the general context of computer executable instructions. Generally, computer executable instructions can include routines, programs, objects, components, data structures, procedures, modules, and functions, which perform specific functions or implement specific abstract data types.

The order in which the method 300 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block 302, respiratory sound signals 112 and user input data are received. In an embodiment, the RMD 102 receives the plurality of respiratory sound signals 112 captured by the acoustic sensors 104. The RMD 102 also receives the user input data 116 from the user interface 114 of the user device 106. In one embodiment, the acoustic sensors 104 may be coupled with the user device 106. For example, the respiratory sound signals 112 i.e. any of cough sound signals, wheeze sound signals and breathing sound signals are captured by the microphone or audio recorder of the mobile computing device and are sent to the RMD 102 coupled with the mobile computing device via communication network. In one example, the user i.e. the person or the patient inputs the user input data 116 comprising the information related to the generic symptoms including body temperature of the person or the patient i.e. fever, Fatigue, Loss of Appetite and the chest related symptoms including Shortness of breath (SOB), Chest Pain, Hemoptysis and other related symptoms as illustrated in FIG. 3b. Based on the received respiratory sound signals 112, the characteristic derivation module 122 derives the primary respiratory sound characteristics 208.

At block 304, the primary respiratory sound characteristics 208 of one or more respiratory sound signals 112 is derived. In an embodiment, the characteristics determination module 122 receives the captured respiratory sound signals 112 and derives the primary respiratory sound characteristics 208 associated with each respiratory sound signal 112. For example, the respiratory sound signals 112 captured by the acoustic sensors 104 are received and the respiratory sound signals 112 likely related to respiratory events i.e. cough events or wheeze events are extracted eliminating the noisy background sound signals and other unwanted sound signals from the captured signals of sound recordings. Upon extracting the respiratory sound signals 112 related to respiratory events, the characteristics determination module 122 derives the primary respiratory sound characteristics 208 including Pitch of the respiratory sound signal, Log energy, Zero crossings, Mel-frequency cepstral coefficients (MFCC) and Formant frequencies of respiratory sound signal. Based on the derived primary respiratory sound characteristics 208, one or more of the secondary respiratory sound characteristics are determined.

At block 306, the secondary respiratory sound characteristics are determined. In an embodiment, the characteristics determination module 122 determines one or more of secondary respiratory sound characteristics from the derived primary respiratory sound characteristics 208. In one example, the secondary respiratory sound characteristics include type 209 of respiratory sound signal 112 and intensity 212 of respiratory sound signal 112 that are determined based on the derived primary respiratory sound characteristics 208. Also, the secondary respiratory sound characteristics further include frequency of occurrence 210 of the respiratory sound signal 112 and the duration 211 of respiratory sound signal 112. For example, if the respiratory sound signal 112 is the cough sound signal then the type 209 of cough sound signal may include one of dry cough signal and the wet cough signal.

In one embodiment, if the respiratory sound signal 112 is the wheeze sound signal then the type 209 of wheeze sound signal may include one of monophonic wheeze sound signal and polyphonic wheeze sound signal. The type 209 of respiratory sound signal 112 is determined based on one or more of Pitch, Log energy, Zero crossings, Mel-frequency cepstral coefficients (MFCC) and Formant frequencies of the respiratory sound signal 112. In one example, the intensity 212 of the respiratory sound signal 112 is determined based on the Pitch and log energy of the primary respiratory sound characteristics 208 of the respiratory sound signals 112. Further, the frequency of occurrence 210 is determined as number of respiratory sound signals 112 occurring in specified time period. For example, if the respiratory sound signal 112 is a cough sound signal, the frequency of occurrence 210 is determined as the number of cough sound signals per minute. Further, the duration 211 is determined as the length of time that the respiratory sound signal 112 persists. For example, in case of cough sound signal, if there exist multiple cough sound signals i.e. a cough sequence for given cough event, the duration is determined as the average duration of the cough event. Based on the determined respiratory sound characteristics, presence of inflammation in one or more of airway part, pleura and parenchyma is determined.

At block 308, the presence of inflammation in one or more of respiratory parts is determined. In one embodiment, the risk assignment module 124 determines the presence of inflammation in one or more of smaller airway, larger airway, pleura and parenchyma based on the secondary respiratory sound characteristics. In one example, for the cough sound signal the presence of inflammation is determined based on the secondary respiratory sound characteristics of the cough sound signal including type 209 Frequency of occurrence 210, Intensity 212 and duration 211, and the primary respiratory sound characteristic 208 of formant frequencies. In one embodiment, the presence of inflammation may also be negligible in one or more of smaller airway, larger airway, pleura and parenchyma. For example, Table 1 gives an indication of how the variation in duration, intensity and frequency of occurrences influence the presence of inflammation. In one embodiment, the presence of inflammation may be indicated by visualization of respiratory sound signals 112 with a highlighted marking using suitable device integrated with RMD 102, wherein the highlighted marking indicates the possible inflammations. For example, RMD 102 may extract valid respiratory sounds like cough sounds, may process, display and print them on a paper with markings highlighting the beginning and end of cough sequences, within each sequence providing indicators to help in visualizing the possible inflammations, presence of mucus etc.

TABLE 1

| | | Freq of occurrence | |
|---|---|---|---|
| Dura- | Inten- | of Cough sounds | |
| tion | sity | (no. of events/ | |
| (in Sec) | (AU) | minute) | Presence of Inflammation |
| 3 | 68 | 6 to 10 | Inflammation in parenchyma. |
| 2 | 81 | 4 to 6 | Inflammation in pleura. |
| 1.04 | 399 | 10 to 12 | Inflammation in Smaller airways. |
| 0.9 | 370 | 10 to 12 | Inflammation in larger airways. |

At block 310, the risk category 213 associated with one or more respiratory sound signals 112 is determined. In an embodiment, the risk assignment module 124 assigns risk associated with captured respiratory sound signals 112 based on the determined presence of inflammation and the user input data 116. In one example, the risk assignment module 124 receives the user input data 116 from the user as illustrated in FIG. 3b and the determined presence of inflammation. The user inputted information related to generic symptoms including body temperature or fever, fatigue and chest related symptoms including shortness of breath, chest pain, hemoptysis and other related symptoms is analyzed considering the determined presence of inflammation as illustrated in FIG. 3c. For example, if there is an inflammation in one of smaller airway, larger airway, pleura and parenchyma, the risk assignment module 124 assigns the risk category 213 including one of high risk, moderate risk and low risk based on the received user input data 116. If the inflammation is not present in all of the parts of respiratory system, the risk assignment module 214 assigns negligible risk to the respiratory sound signal 112. For example, based on the user input data given by the user as illustrated in FIG. 3b and the determined presence of inflammation, the risk assignment module 124 assigns a high-risk category as illustrated in FIG. 3d. Thus, the present disclosure performs accurate risk analysis on the respiratory sound signals 112 and effectively analyzes the risk associated with respiratory sounds.

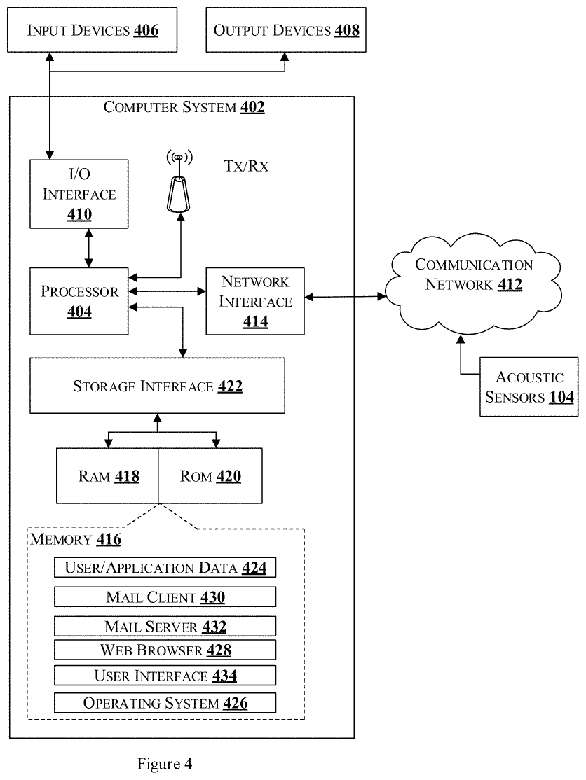

FIG. 4 is a block diagram of an exemplary computer system for implementing embodiments consistent with the present disclosure.

In an embodiment, the computer system 402 may be risk monitoring device 102, which is used for analyzing risk associated with respiratory sounds. The computer system 402 may include a central processing unit (“CPU” or “processor”) 404. The processor 404 may comprise at least one data processor for executing program components for executing user or system-generated business processes. The processor 404 may include specialized processing units such as integrated system (bus) controllers, memory management control units, floating point units, graphics processing units, digital signal processing units, etc.

The processor 404 may be disposed in communication with one or more input/output (UO) devices (406 and 408) via I/O interface 410. The I/O interface 410 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc.

Using the I/O interface 410, the computer system 402 may communicate with one or more I/O devices (406 and 408). In some implementations, the processor 404 may be disposed in communication with a communication network 412 via a network interface 414. The network interface 414 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. Using the network interface 414 and the communication network 412, the computer system 402 may be connected to the acoustic sensors 104 for receiving one or more respiratory sound signals captured by one or more acoustic sensors 104-1, 104-2, . . . , 104-N.

The communication network 412 can be implemented as one of the several types of networks, such as intranet or any such wireless network interfaces. The communication network 412 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 412 may include a variety of network devices, including routers, bridges, servers, computing devices, storage devices, etc.

In some embodiments, the processor 404 may be disposed in communication with a memory 416 e.g., RAM 418, and ROM 420, etc. as shown in FIG. 4, via a storage interface 422. The storage interface 422 may connect to memory 416 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 416 may store a collection of program or database components, including, without limitation, user/application 424, an operating system 426, a web browser 428, a mail client 430, a mail server 432, a user interface 434, and the like. In some embodiments, computer system 402 may store user/application data 424, such as the data, variables, records, etc. as described in this invention. Such databases may be implemented as fault-tolerant, relational, scalable, secure databases such as Oracle or Sybase.

The operating system 426 may facilitate resource management and operation of the computer system 402. Examples of operating systems include, without limitation, Apple Macintosh OS X, UNIX, Unix-like system distributions (e.g., Berkeley Software Distribution (BSD), FreeBSD, Net BSD, Open BSD, etc.), Linux distributions (e.g., Red Hat, Ubuntu, K-Ubuntu, etc.), International Business Machines (IBM) OS/2, Microsoft Windows (XP, Vista/7/8, etc.), Apple iOS, Google Android, Blackberry Operating System (OS), or the like. A user interface may facilitate display, execution, interaction, manipulation, or operation of program components through textual or graphical facilities. For example, user interfaces may provide computer interaction interface elements on a display system operatively connected to the computer system 402, such as cursors, icons, check boxes, menus, windows, widgets, etc. Graphical User Interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

While various aspects and embodiments have been disclosed herein, other aspects and embodiments will be apparent to those skilled in the art.

1. A method for analyzing risk associated with respiratory sounds, method comprising:
receiving, by a processor of a respiratory monitoring device, a plurality of respiratory sound signals captured by at least one acoustic sensor, and a user input data from a user interface of a user device coupled with the respiratory monitoring system;
deriving, by the processor, one or more primary respiratory sound characteristics including Pitch, Log energy, Zero crossings, Mel-frequency cepstral coefficients (MFCC) and Formant frequencies for each captured respiratory sound signal; and
assigning, by the processor, a risk category to the plurality of respiratory sound signals based on the derived primary respiratory sound characteristics and the user input data.

2. The method as claimed in claim 1, wherein the user input data comprises information associated with chest related symptoms including Shortness of Breath, chest pain, Hemoptysis and other generic symptoms including body temperature of the person or the patient i.e. fever, Fatigue, Loss of Appetite.

3. The method as claimed in claim 1, wherein the respiratory sound signals is one or more of cough sound signals, wheeze sound signals and breathing sound signals.

4. The method as claimed in claim 1, wherein the step of assigning the risk category comprising steps of:
determining one or more secondary respiratory sound characteristics for each respiratory sound signal based on the primary respiratory sound characteristics;
determining presence of inflammation in one or more of smaller airway, larger airway, parenchyma and pleura based on the determined secondary respiratory sound characteristics; and
assigning the risk category associated with the captured respiratory sound signals based on the presence of inflammation and the user input data.

5. The method as claimed in claim 4, wherein the secondary respiratory sound characteristics include one or more of a frequency of occurrence, a duration, an average intensity and a type of respiratory sound signals.

6. The method as claimed in claim 4, wherein the risk category assigned to the respiratory sound signals is one of negligible, low, moderate and high-risk category.

7. A system for analyzing risk associated with respiratory sounds, system comprising:
at least one acoustic sensor for capturing a plurality of respiratory sound signals;
a user device coupled with the at least one acoustic sensor comprising a user interface for receiving a user input data from at least one user, and capable of transmitting the captured respiratory sound signals and the user input data to a respiratory monitoring device; and
the respiratory monitoring device (RMD) communicatively coupled with the user device via a communication network, wherein the RMD comprises a processor configured to:
receive the captured plurality of respiratory sound signals and the user input data;
derive one or more primary respiratory sound characteristics including Pitch, Log energy, Zero crossings, Mel-frequency cepstral coefficients (MFCC) and Formant frequencies for each captured respiratory sound signal; and
assign a risk category to the plurality of respiratory sound signals based on the derived primary respiratory sound characteristics and the user input data.

8. The system as claimed in claim 7, wherein the user input data comprises information associated with chest related symptoms including Shortness of Breath, chest pain, Hemoptysis and other generic symptoms including body temperature of the person or the patient i.e. fever, Fatigue, Loss of Appetite.

9. The system as claimed in claim 7, wherein the respiratory sound signals is one or more of cough sound signals, wheeze sound signals and breathing sound signals.

10. The system as claimed in claim 7, wherein the processor is configured to assign a risk category by:
determining one or more secondary respiratory sound characteristics for each respiratory sound signal based on the primary respiratory sound characteristics;
determining presence of inflammation in one or more of smaller airway, larger airway, parenchyma and pleura based on the determined secondary respiratory sound characteristics; and
assigning the risk category associated with the captured respiratory sound signal based on the presence of inflammation and the user input data.

11. The system as claimed in claim 10, wherein the secondary respiratory sound characteristics includes one or more of a frequency of occurrence, a duration, an average intensity and a type of respiratory sound signals.

12. The system as claimed in claim 10, wherein the risk category assigned to the respiratory sound signals is one of negligible, low, moderate and high risk category.

13. The system as claimed in claim 7, further comprises a data repository for storing the captured respiratory sound signals and the user input data.

14. The method as claimed in claim 5, wherein the determining presence of inflammation in the parenchyma comprises determining that one or more frequency of occurrence of the cough from 6 to 10 events per minute.

15. The method as claimed in claim 5, wherein the determining presence of inflammation in the pluera comprises determining that one or more frequency of occurrence of the cough from 4 to 6 events per minute.

16. The method as claimed in claim 5, wherein the determining presence of inflammation in the smaller airways comprises determining that one or more frequency of occurrence of the cough from 10 to 12 events per minute.

17. The system as claimed in claim 11, wherein the determining presence of inflammation in the parenchyma comprises determining that one or more frequency of occurrence of the cough from 6 to 10 events per minute.

18. The method as claimed in claim 11, wherein the determining presence of inflammation in the pluera comprises determining that one or more frequency of occurrence of the cough from 4 to 6 events per minute.

19. The method as claimed in claim 11, wherein the determining presence of inflammation in the smaller airways comprises determining that one or more frequency of occurrence of the cough from 10 to 12 events per minute.
